(19) United States
(12) Patent Application Publication
(10) Pub. No.: US 2025/0160692 A1
(43) Pub. Date: May 22, 2025

(54) POLYESTERAMIDE COPOLYMERS FOR MEMBRANES AND COATINGS FOR GLUCOSE SENSORS

(71) Applicant: DSM IP Assets B.V., Heerlen (NL)

(72) Inventors: Aylvin Jorge Angelo Athanasius DIAS, ECHT (NL); Inge Jeannette MINTEN, ECHT (NL); Meredith Elsa WISEMAN, ECHT (NL)

(21) Appl. No.: 18/839,913

(22) PCT Filed: Feb. 24, 2023
(86) PCT No.: PCT/EP2023/054638

§ 371 Date: Aug. 20, 2024

Related U.S. Application Data
(60) Provisional application No. 63/313,584, filed on Feb. 24, 2022.

(51) Int. Cl. A61B 5/145; A61B 5/1486; C08G 69/44

(52) U.S. Cl. CPC A61B 5/14532; A61B 5/1451; A61B 5/14865; C08G 69/44

(57) ABSTRACT

Polyesteramide (PEA) copolymers are disclosed, having structures that include repeating units that contain various monomer residues characterized herein and present in the repeating units at defined ranges of molar equivalents. These copolymers can possess a number of important advantages for providing longer performing glucose sensors and/or for simplifying their design. For example, in vivo modeling has confirmed that these polymers exhibit both decreased collagen deposition and increased neovascularization (angiogenesis). Additional testing has demonstrated favorable oxygen and glucose transport capabilities, as well as other relevant properties.

### CROSS-REFERENCE TO RELATED APPLICATIONS

This application is an international application claiming priority to U.S. Provisional Patent Application No. 63/313,584, filed 24 Feb. 2022, the entire contents of which is hereby incorporated by reference in its entirety.

### FIELD OF THE INVENTION

Aspects of the invention relate to polyesteramide polymers and more particularly those exhibiting advantageous in vivo performance characteristics as well as glucose and oxygen permeability and other properties that render the polyesteramide polymers suitable, according to specific embodiments, for use as membranes and coatings in continuous glucose monitoring (CGM), such as for implantable glucose sensors.

### DESCRIPTION OF RELATED ART

Ensuring timely and accurate measurements of blood glucose levels is important to allow users of glucose monitors to react and prevent further complications that may arise from diabetes. Continuous glucose sensors of glucose monitoring systems typically have a limited in situ use life of up to only 10 days. Factors contributing to this short lifetime include fibrosis and excess collagen deposition, as well as poor vascularization in the immediate vicinity of the device. As evidence of these phenomena, a recent study by McClatchey et al. (DIABETES 2019; 68(10):1892-1901) confirmed that fibrotic encapsulation is the main source of deterioration of the glucose sensor, and more specifically loss of sensor function can be closely correlated with collagen content. With respect to fibrosis induced vessel regression, Klueh et al. (J BIOMED MATER RES PART A 2014:102A:3512-3522) have postulated that stimulation of neovascularization could offset this condition and enhance both the function and lifespan of a glucose sensor.

Approaches to prolonging the lifetime of glucose sensors have involved the use of drugs or biologically active species to inhibit the foreign body response (FBR) and its detrimental effects as described above. Such agents, however, not only complicate sensor fabrication but also significantly increase the challenge of the regulatory pathway to approval. For example, a dexamethasone eluting coating has been attempted, but the long-term use of this and other steroidal anti-inflammatory agents is undesirable. In addition, drug release should be correlated with in vivo measurements, as well as carefully controlled to ensure that sensor function is not impaired. Other biomaterials-based approaches, for example as described in U.S. Pat. No. 9,579,422, have involved the use of collagen coatings on sensors to promote biocompatibility and neovascularization (angiogenesis). The use of animal derived collagen, and generally the intrinsic variability of bio-derived collagen, presents its own set of considerable challenges to producing a consistently performing sensor.

A reduction in sensor life, due to the effects described above, translates to a need for regular and timely replacement of the sensor, in addition to enhanced user attention and compliance. In this regard, it is well recognized that in the realm of self-administered medication and implanted sensors, patient compliance is a significant challenge. There remains a need in the art, therefore, for improvements in glucose monitors and their sensor components that provide a satisfactory solution for prolonging their useful operating life.

### SUMMARY OF THE INVENTION

Aspects of the invention are associated with the discovery of polyesteramide (PEA) polymers that, compared to conventional polymers for use in continuous glucose monitoring (CGM), exhibit surprisingly beneficial properties for this purpose. This has been demonstrated, for example, using a mouse model in which an implant window was coated with a PEA polymer of interest and tested against control polymers and a porcine punch biopsy study wherein PEA coated filaments are compared to filaments from a commercial glucose sensor. It was revealed that PEA polymers are good candidates for providing longer performing glucose sensors, in view of both decreased collagen deposition and increased neovascularization (angiogenesis). Overall, these polymers perform well in controlling the foreign body response (FBR) that is initiated upon implantation of a device and that is associated with inflammation and excess collagen deposition. The FBR typically interferes with the operation of glucose sensors, due to adverse effects on transport properties in the immediate microenvironment of the sensor component. Reduction in glucose transport and decreased sensor function leads to a drift in sensor signaling that requires in silico algorithmic correction to compensate for signal loss. However, this ability to compensate has its limits, and eventually the sensor must be replaced.

In addition to exhibiting favorable in vivo performance with respect to the FBR that can prolong the life of glucose sensors, PEA polymers as described herein have been demonstrated to possess other properties that are important for a glucose limiting membrane, and particularly those used in CGM. In various embodiments, these properties can include acceptability in terms of one or more of oxygen to glucose permeability ratio, glucose permeability, glass transition temperature, lack of crystallinity, water uptake characteristics, and shear storage modulus. According to some embodiments, PEA polymers as described herein, as well as membranes and coatings comprising these polymers, are not required to carry an anti-inflammatory agent to favorably inhibit the FBR. This is surprising in view of known uses of PEA polymers, for example as described in WO 2014/096339, for applications of drug delivery. Also, PEA polymers as described herein exhibit favorable characteristics in terms of adhesion to relevant parts of a glucose sensor, which include their needle like components and infusion cannula, particularly compared to the alternative proposed solution of a collagen coating. Further advantages reside in these PEA polymers being surface eroding as opposed to being biodegradable generally. This property renders the PEA polymers particularly suitable in embodiments in which they are used as external membrane coatings.

Some embodiments are directed to a PEA polymer (also referred to herein more simply as a PEA) having a structure including units of monomer residues, as defined below, that are present in these units at respective molar equivalents that are designated by m, p, q, and x, in which m+p+q+x=1. To the extent that respective values of m, p, q, and/or x may be 0 in any general or more specific embodiments as defined herein, the associated monomer residues may be absent in such embodiments. Other embodiments are directed to glucose limiting membranes comprising these PEA polymers and optionally possessing one or more of the properties described above, and as elaborated on in more detail below, which are important for such membranes. Yet other embodiments are directed to coatings for implantable glucose sensors, the coatings comprising PEA polymers having a structure as defined herein and/or possessing one or more of the properties described above, and as elaborated on in more detail below.

In more particular embodiments, such membranes or coatings do not include an anti-inflammatory agent (i.e., an anti-inflammatory agent is absent from a representative membrane or coating), or do not include more specifically a steroidal anti-inflammatory agent (e.g., do not include dexamethasone). In other more particular embodiments, such membranes or coatings may comprise an additional polymer, such as a non-biodegradable (biostable) polymer. An exemplary polymer in this regard is a biostable polyurea or polyurethane polymer. A polyurethane may optionally include polycarbonate and/or siloxane groups, and/or may be blended with a hydrophilic polymer such as polyvinylpyrrolidone. According to one configuration, the additional (e.g., biostable) polymer may underlie an external surface layer of the PEA polymer, with the external surface layer being configured for direct contact with interstitial fluid of the patient. In this manner, the PEA may serve as a biodegradable and surface erodible (or surface eroding) component of the membrane or coating.

Yet other embodiments are directed to glucose sensors (e.g., implantable glucose sensors) having such membranes or coatings applied to components thereof, for example those for penetrating the skin of the patient (e.g., configured to be positioned subcutaneously during use, for interacting with and/or for sampling of interstitial fluid of the patient). Representative components include needle-like structures or insulin infusion cannula. In the case of an implantable glucose sensor having such membrane or coating, still further embodiments are directed to methods for continuously monitoring a glucose level in a patient. The methods comprise implanting the implantable glucose sensor in the patient (e.g., with the implanting being performed by the patient himself/herself), such that glucose and oxygen in interstitial fluid of the patient pass through the membrane or coating, thereby allowing the sensor to detect and/or monitor a glucose level in the patient.

These and other embodiments, aspects, and advantages relating to the present invention are apparent from the following Detailed Description.

### BRIEF DESCRIPTION OF THE DRAWINGS

A more complete understanding of exemplary embodiments of the invention and the advantages thereof may be acquired by referring to the following description in consideration of the accompanying figures, which serve to illustrate various features and certain principles involved.

FIG. 1 illustrates a general reaction scheme for preparing the random copolymer designated PEA III AcBz.

FIG. 2 provides a bar graph of white fraction data obtained with the PEA and PLGA coatings, corresponding to collagen deposition, following a 14-day experimental period in which PEA- or PLGA-coated windows were implanted in mice, as described below.

### DETAILED DESCRIPTION

Glucose limiting membranes are glucose and oxygen permeable structures that may be used in continuous glucose monitoring (CGM). Embodiments of the invention are directed to glucose limiting membranes comprising a polyesteramide (PEA), which may be characterized as a random copolymer. The PEA has a structure that includes, or that may consist of, n units, each unit having monomer residues, for example 2, 3, or 4 different types of monomer residues, as defined below, which are present in each unit at respective molar equivalents that are designated by m, p, q, and x, in which m+p+q+x=1. The n units may be characterized as “repeating” units to the extent that the defined values or ranges of m, p, q, and x are consistent from one unit to the next, although it is not required for each unit to be identical in structure, as long as the defined values or ranges are met. As is apparent from the structures of monomer residues given below, they are linked by amide (peptide) bonds [(—C═O)NH—)], whereas certain of these residues include ester bonds [(—C═O)O—)] that help confer water permeability. Given the teachings herein, those skilled in the art will be able to appreciate the structural modifications and corresponding adjustments in synthesis procedures that influence glucose and oxygen permeability, as well as other properties described herein.

For ease of understanding, dashed lines are used in structures of the individual monomer residues, and in other structures, to emphasize that they represent points of attachment (bonding) and not methyl groups. To the extent that structures may include chiral carbon atoms, the lack of an express illustration of stereochemistry is meant to convey that all stereoisomers are intended. However, those skilled in organic chemistry, given the teachings herein including the specific synthesis procedures, will be able to ascertain particular stereochemical configurations that are representative of PEA polymers in preferred embodiments.

More specifically, the n units in the in the structure of the PEA may have:
m molar equivalents of a first monomer residue having the formula

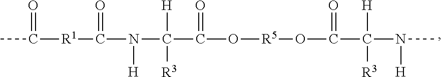

p molar equivalents of a second monomer residue having the formula

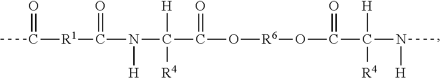

q molar equivalents of a third monomer residue having the formula

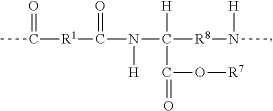

and
x molar equivalents of a fourth monomer residue having the formula

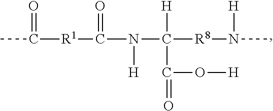

The values m, p, q, and x represent molar equivalents of the corresponding first, second, third, and fourth monomer residues in the units of the random copolymer, which molar equivalents are scaled such that m+p+q+x=1, thereby indicating relative molar equivalents among the monomer residues.

According to a first embodiment, m is from 0 to 0.8, p is from 0 to 0.95, m+p is from is from 0.5 to 1, q is from 0 to 0.35, and x is from 0 to 0.25; n is from 5 to 300; R¹ is (C₂-C₂₀) alkylene; R³ and R⁴ are selected from the group consisting of hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃⁺, —(CH₂)₃NHC(═NH₂⁺)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph—CH₂—, CH═C—CH₂—, (CH₃)₂CH—, Ph—NH—, NH—(CH₂)₃—C—, or NH—CH═N—CH═C—CH₂—, wherein R³ and R⁴ are the same or different; R⁵ is (C₂-C₂₀)alkylene, optionally having one or more alkylene carbon atoms (—C—) replaced by an oxygen atom (—O—), and/or optionally having 5 or 6 consecutive alkylene carbon atoms together form a respective 5- or 6-membered carbocyclic ring;
R⁶ is

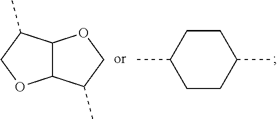

R⁷ is (C₆-C₁₀)aryl(C₁-C₆)alkylene; and R⁸ is (C₃-C₈)alkylene. In such PEA polymers, higher values of p, i.e., increased proportions of the second monomer residue, can lead to a higher glass transition temperature, and more particularly a higher glass transition temperature of the polymer in its hydrated state (wet Tg).

As used herein, the term “alkyl” means a monovalent straight or branched chain hydrocarbon group including methyl, ethyl, n-propyl, isopropyl, n-butyl, isobutyl, tert-butyl, n-hexyl, and the like, with more specific alkyl groups being limited according to ranges of carbon atom numbers, as indicated in parentheses, preceding “alkyl.”

As used herein, the term “alkylene” means a divalent straight or branched chain hydrocarbon group such as —CH₂—, —(CH₂)₂—, —(CH₂)₃—, —(CH₂)₄—, —(CH₂)₅—, and the like, with more specific alkyl groups being limited according to ranges of carbon atom numbers, as indicated in parentheses, preceding “alkylene.”

As used herein, the term “alkenyl” means a monovalent straight or branched chain hydrocarbon group having at least one carbon-carbon double bond in the main chain or in a side chain, with more specific alkenyl groups being limited according to ranges of carbon atom numbers, as indicated in parentheses, preceding “alkenyl.”

As used herein, the term “alkenylene” means a divalent straight or branched chain hydrocarbon group having at least one carbon-carbon double bond in the main chain or in a side chain, with more specific alkenylene groups being limited according to ranges of carbon atom numbers, as indicated in parentheses, preceding “alkenylene.”

As used herein, “alkynyl” means a monovalent straight or branched chain hydrocarbon group having at least one carbon-carbon triple bond in the main chain or in a side chain, with more specific alkynyl groups being limited according to ranges of carbon atom numbers, as indicated in parentheses, preceding “alkynyl.”

As used herein, “aryl” means an unsubstituted or optionally substituted phenyl radical or an unsubstituted or optionally substituted ortho-fused bicyclic carbocyclic radical having nine or ten ring atoms, in which at least one ring is aromatic. Examples of aryl include, but are not limited to, phenyl, naphthyl, and nitrophenyl.

As used herein, “biodegradable” means a material which is capable of being completely or substantially degraded or eroded when exposed to an in vivo environment. A polymer is capable of being degraded or eroded when it can be gradually broken down, resorbed, absorbed, and/or eliminated by, for example, hydrolysis, enzymolysis, oxidation, metabolic processes, bulk or surface erosion, and the like. As noted above, in preferred embodiments, PEA polymers may be characterized as being biodegradable and more particularly surface erodible (or surface eroding), meaning that bio-degradation results selectively from surface erosion. This leads to advantages in the use of such polymers in glucose limiting membranes.

According to the PEA structure as defined above and including n units, each having monomer residues, for example 2, 3, or 4 different types of monomer residues, in the case of a “random copolymer,” the monomer residues, present in each unit at respective molar equivalents that are designated by m, p, q, and x, are distributed randomly throughout the units and throughout the copolymer.

Given the structures of the monomers above, it can be appreciated that, in any of the embodiments as defined herein, the PEA may have the following structure:

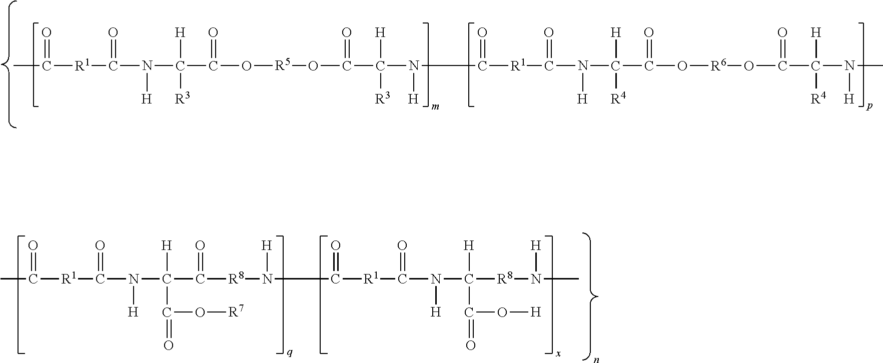

wherein n, m, p, q, and x, as well as the substituents R¹, R³, R⁴, R⁵, R⁶, R⁷, and R⁸ are as defined above.

In more specific embodiments, the PEA is defined as according to the first embodiment, wherein m may be from 0.10, 0.15, 0.20, or 0.25 to 0.75, 0.70, 0.65, 0.60, 0.55, 0.50, 0.45, 0.40, or 0.35. In other more specific embodiments, the PEA is defined as according to the first embodiment, wherein p may be from 0.10, 0.20, 0.30, 0.35, or 0.40 to 0.90, 0.85, 0.75, 0.70, 0.65, 0.60, 0.55, 0.50, or 0.45. In other more specific embodiments, the PEA is defined as according to the first embodiment, wherein p may be greater than or equal to m. In other more specific embodiments, the PEA is defined as according to the first embodiment, wherein m and p may be both greater than zero. In other more specific embodiments, the PEA is defined as according to the first embodiment, wherein m:p may be from 2:1, 1:1, or 2:3 to 1:5, 1:4, 1:3, or 1:2. In other more specific embodiments, the PEA is defined as according to the first embodiment, wherein q is from 0.05, 0.10, 0.12, 0.14, 0.15, 0.16, 0.17, 0.18, 0.19, or 0.2 to 0.30, 0.25, 0.23, 0.21, 0.20, 0.19, 0.18, 0.17, 0.16, or 0.15. In other more specific embodiments, the PEA is defined as according to the first embodiment, wherein x is from 0.05, 0.06, 0.07, 0.08, 0.09, or 0.10 to 0.25, 0.20, 0.15, 0.14, 0.13, 0.12, 0.11, or 0.10. In other more specific embodiments, the PEA is defined as according to the first embodiment, wherein the ratio q:x is from 9:1, 8:1, 7:1, 6:1, 5:1, 4:1 or 3:1 to 1:4, 1:3, 1:2, 1:1, 2:1, or 3:1. In other more specific embodiments, the PEA is defined as according to the first embodiment, wherein m is about 0.3, p is about 0.45, q is about 0.19, and x is about 0.06. In other more specific embodiments, the PEA is defined as according to the first embodiment, wherein n is from 5, 10, 15, 20, 25, 30, 35, 40, 45, 50, 55, 60, 65, 70, or 75 to 300, 290, 280, 270, 260, 250, 240, 230, 220, 210, 200, 190, 180, 170, 160, or 150. In other more specific embodiments, the PEA is defined as according to the first embodiment and has a number average molecular weight (Mn) of at least 15,000 g/mol, at least 20,000 g/mol, at least 25,000 g/mol, at least 30,000 g/mol, or at least 35,000 g/mol. In other more specific embodiments, the PEA is defined as according to the first embodiment and has an Mn of at most 250,000 g/mol, at most 225,000 g/mol, at most 200,000 g/mol, at most 175,000 g/mol, at most 150,000 g/mol, at most 125,000 g/mol, at most 100,000 g/mol, or at most 75,000 g/mol. Mn is measured via gel permeation chromatography (GPC) in tetrahydrofuran (THF) with polystyrene as standard.

In yet further specific embodiments, the PEA is defined as according to the first embodiment, and may have any one or more of: ranges of m as defined above; ranges of p as defined above; p being greater than m; m and p being both greater than zero; ranges of m:p as defined above; ranges of q as defined above; ranges of x as defined above; ranges of q:x as defined above; values of m, p, q, and x as defined above; ranges of n as defined above; lower limits of Mn as defined above; and/or upper limits of Mn as defined above.

In yet further specific embodiments, the PEA is defined as according to the first embodiment, and may be more particularly characterized by any one or more of: R³ is selected from the group consisting of hydrogen, (C₁-C₆)alkyl, CH₃—CH₂—CH(CH₃)—, (CH₃)₂CH—CH₂—, Ph—CH₂—, and (CH₃)₂CH—; R⁴ is selected from the group consisting of hydrogen, (C₁-C₆)alkyl, CH₃—CH₂—CH(CH₃)—, (CH₃)₂CH—CH₂—, Ph—CH₂—, and (CH₃)₂CH—; R³ and R⁴ are the same; R⁵ is (C₂-C₂₀)alkylene; R⁷ is (C₆)aryl-CH₂— (i.e. benzyl or phenylmethyl); and/or R⁸ is —(CH₂)₄—.

In yet further specific embodiments, the PEA is defined as according to the first embodiment, and may be more particularly characterized by any one or more of: p is from 0 to 0.8 and m+p is from 0.5 to 0.9; q is from 0.05 to 0.25; x is from 0.05 to 0.25; q:x is from 9:1 to 1:4, or more particularly from 4:1 to 1:4 or from 4:1 to 1:2; q is 0; x is 0; and/or m is 0.

In yet further specific embodiments, the PEA is defined as according to the first embodiment, and may be more particularly characterized by any one or more of: m is from 0.1 to 0.5, p is from 0.1 to 0.75, m+p is from 0.5 to 0.8, q is from 0.1 to 0.35, and x is from 0 to 0.1; R¹ is (C₂-C₁₀)alkylene; R³ and R⁴ are (C₁-C₆)alkyl; R⁵ is (C₂-C₁₀)alkylene;
R⁶ is

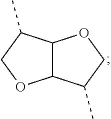

R⁷ is Ph-(C₁-C₆)alkylene; and R⁸ is (C₃-C₆)alkylene. For example, the PEA may be characterized by one or more of these definitions of m, p, m+p, q, x, R¹, R³, R⁴, R⁵, R⁶, R⁷, and R⁸, such as in the case of the PEA being characterized by all of these definitions.

In yet further specific embodiments, the PEA is defined as according to the first embodiment, and may be more particularly characterized by any one or more of: m is 0.3, p is 0.45, q is 0.25, and x is 0; R¹ is octylene [(C₈)alkylene]; R³ and R⁴ are isobutyl; R⁵ is hexylene [(C₆)alkylene];
R⁶ is

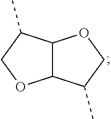

R⁷ is benzyl [Ph-(C₁)alkylene, or phenylmethyl]; and R⁸ is butylene [(C₄)alkylene]. For example, the PEA may be characterized by one or more of these definitions of m, p, q, x, R¹, R³, R⁴, R⁵, R⁶, R⁷, and R⁸, such as in the case of the PEA being characterized by all of these definitions.

Glucose sensors commonly use an immobilized glucose oxidase enzyme which relies on oxygen as a cofactor to generate a signal. As the range of physiological oxygen concentrations is roughly two orders of magnitude lower than the range of physiological glucose concentrations, such sensors rely on a glucose limiting membrane to decrease the local concentration of glucose at the sensor relative to that of oxygen. This allows the sensor to provide a linear response to the concentration of glucose present over the full dynamic range in expected use. One attribute of PEA for use in a glucose sensor therefore relates to its ability to allow transport of both glucose and oxygen, according to defined relative amounts. For effective operation of a glucose sensor, the ratio of oxygen to glucose permeabilities should be close to, and preferably greater than, the ratio of the maximum physiologically expected concentration of glucose to the minimum physiologically expected concentration of oxygen. In view of this, representative glucose limiting membranes and coatings comprising a PEA polymer as described herein, or the PEA polymer itself, may have an oxygen to glucose permeability ratio of at least about 350, at least about 440, or at least about 500. For example, a maximum blood glucose concentration of 22 mM and a minimum blood oxygen concentration of 0.05 mM would lead to the calculated, preferred ratio of at least about 440. To the extent that PEA polymers have been developed for drug delivery and are engineered to allow delivery of small molecule active pharmaceutical ingredients (APIs), there is no basis to predict from this, whether or not PEA polymers would exhibit favorable oxygen to glucose permeability ratios for use in glucose sensors. The fact that such ratios can be achieved, as disclosed herein, is considered surprising.

In addition, it is believed that there is also a lower practical limit of glucose permeability for a glucose limiting membrane, regardless of whether any particular oxygen to glucose permeability ratio criterion is met. If the glucose permeability is too low, the amount of glucose reaching the enzyme layer of the sensor will likewise be too low for a sufficiently strong signal. Accordingly, representative glucose limiting membranes or coatings comprising a PEA polymer as described herein, or the PEA polymer itself, may have a glucose permeability of at least about 10⁻¹⁵ cm²/s, or more particularly at least about 10⁻¹² cm²/s (based on a partition coefficient of unity). A practical, alternative indicator for meeting a lower bound of glucose permeability is the glass transition temperature of the PEA in the hydrated state (wet Tg). If the wet Tg is substantially greater than body temperature (about 37° C., or possibly down to about 34° C. in the skin layers), the PEA polymer will be glassy and therefore less amenable for allowing passage of small molecules such as glucose or oxygen. Accordingly, the PEA polymer of representative glucose limiting membranes or coatings as described herein may have a wet Tg of less than about 40° C., less than about 37° C., less than about 34° C., or possibly less than about 30° C. Wet Tg can be measured in a rheometer by performing a temperature ramp test at a fixed frequency and determining the temperature at which the loss modulus (G″) reaches a local maximum. In view of similar considerations, an amorphous PEA may be preferable over a semicrystalline PEA, with the amorphous nature being determined by differential scanning calorimetry (DSC) that reveals a substantial absence of melting transitions.

In should be noted that there are 3 generations of glucose biosensors based on different mechanisms of electron transfer, including the use of natural secondary substrates, artificial redox mediators, or direct electron transfer, In the first-generation glucose biosensors, glucose is decomposed catalytically via glucose oxidase (Gox) and the H2O2 is generated and then subsequently oxidized at the electrode surface, producing a measurable current signal. The first-generation glucose biosensor relies on the use of the oxygen as the physiological electron acceptor. They are subject to errors resulting from depletion in local oxygen concentration. The second-generation of glucose biosensors have been proposed for addressing this oxygen limitation. Artificial electron mediators (M), for example, ferro/ferricyanide, hydroquinone, ferrocene, and various redox organic dyes between the electrode and the GOx are employed. These mediators make the electron transfer rate between the electrode and the GOx faster and also offer improvements for when limited oxygen pressure is present. In the third-generation glucose biosensor, the GOx is directly coupled to the electrode. The direct electron transfer efficiently generates an amperometric output signal. The improved sensing performance by the direct electron transfer has been realized by incorporating the enzyme with metal nanoparticles and semiconductive nanomaterials. PEA coatings can also be used for Gen 2 and Gen3 sensors where there is no automatic requirement for oxygen transport but there remains a need for good glucose transport.

Another property of interest is the water uptake of the PEA. Water can act as a plasticizer for the PEA and thereby affect its transport properties. Since the sensor requires calibration according to these transport properties, it is preferable that the transport properties of the PEA do not change substantially over the course of the sensor lifetime, typically on the order of up to 14 days. Water uptake can be assessed gravimetrically, beginning from a dried state of the polymer. In this regard, the PEA polymer of representative glucose limiting membranes and coatings as described herein may have a water uptake of less than about 30 wt-%, less than about 25 wt-%, less than about 20 wt-%, less than about 15 wt-%, less than about 10 wt-%, or less than about 5 wt-%, after two weeks in phosphate buffered saline (PBS) at 37° C. to simulate physiological conditions. The same determination of water uptake on an absolute basis can be used to evaluate the stability of water uptake over a given period, representative of a period of use, for example following an initial, transient period with regard to water uptake. According to some embodiments, the PEA polymer of representative glucose limiting membranes and coatings as described herein may have a water uptake that remains within about +/−25%, within about +/−20%, within about +/−15%, or within about +/−10%, over a period from 4 days to two weeks in PBS at 37° C.

Further with respect to mechanical properties, if the dry Tg of the PEA is too low, the polymer may have a rubbery consistency at room temperature, rendering it so soft as to become sticky or tacky. This could result in undesirable adherence of the sensor to its packaging, and/or create barriers to insertion of the sensor needle. A useful characterization of material tackiness can be made according to the Dahlquist criterion, which characterizes an adhesive based on whether it is soft enough to be deformed by surface tension and thus able to make intimate contact with rough surfaces under minimal contact pressure. A quantity that relates to this criterion is the shear storage modulus at a relevant frequency. In particular, in the dry state at 37° C., the PEA polymer of representative glucose limiting membranes and coatings as described herein may have a shear storage modulus G′ at a frequency of 1 Hz of at least about 0.1 MPa, and more preferably at least about 1 MPa. The shear storage modulus can be measured in the same manner as Tg in a rheometer, by performing a temperature ramp test at the fixed frequency. Alternatively, the Young's modulus can be measured in a tensile mode (DMTA), and the shear modulus can be estimated according to:

$$G = \frac{E}{2 ( 1 + v )} \approx \frac{E}{3} ,$$

according to which E is the Young's modulus and v is Poisson's ratio of the material, commonly about 0.5 for rubbery polymers.

Advantageously, according to preferred embodiments, glucose limiting membranes or coatings described herein, in view of their favorable properties in use that relate to low collagen deposition with good vascularization, may forego the need for addition of drugs or small bioactive molecules to suppress the FBR. Because such agents must elute from the sensor, their elimination simplifies sensor fabrication and the pathway to approval. In addition, it can be appreciated that drug release must be carefully controlled to ensure no interference with sensor function and must also be correlated with in vivo measurements. In general, to the extent that most glucose sensor coatings and membranes rely on a number of transport property requirements such as those relating to glucose and oxygen permeability, the introduction of an active drug to prolong the sensor life adds yet a further degree of design complexity, in addition to the risk of unwanted side reactions attributable to the drug. Furthermore, the drug may interact chemically or physically with the membrane material, detrimentally changing its properties. On this point, the side effects of dexamethasone and other steroidal anti-inflammatory agents are well known from the literature. Avoiding the use of drugs, in the case of representative glucose limiting membranes or coatings described herein, means that application to sensor needles or infusion cannula needles is greatly simplified, since biomaterial solubility in the coating, and not the drug solubility as well, becomes the sole consideration. Yet further advantages reside in the biodegradable PEA of membranes and coatings described herein being surface erodible over time. This can further prolong sensor function as material loss occurs one layer at a time.

Polyesteramide random copolymers may be synthesized by adapting a procedure known in the art from Katsarava et al. (WON J POLYM SCI A: POLYM CHEM 1999:37: 391-407). Briefly, the polymers are prepared via solution polycondensation of di-p-toluenesulfonic or hydrochloric acid salts of bis-(α-amino acid) α,ω-diol diesters, lysine benzyl ester, lysine, and/or di-N-hydroxysuccinimide ester of sebacic acid in anhydrous DMSO. Typically, the salts are converted to free amines by addition of triethylamine and these amines are further reacted with the di-acid derivative. The usage of pre-activated acid in the reaction allows polymerization at relatively low temperature, such as 65° C., affording side-product free polycondensates and predictable degradation products. Subsequently, the obtained reaction mixture is purified via a water precipitation followed by an organic precipitation and filtration. Drying under reduced pressure yields the polyesteramide random copolymer.

For example, such polymers may be prepared by reacting lysine, lysine benzyl ester, and hexahydrofuro[3,2-b]furan-3,6-diyl bis(2-amino-4-methylpentanoate) with di-N-hydroxysuccinimide ester activated sebacic acid in DMSO for 24 hours. The polymer is then isolated from the reaction mixture in two precipitation steps and characterized by means of proton NMR and THF-based GPC relative to polystyrene standards.

### EXAMPLES

The following examples are set forth as representative of the present invention. These examples are not to be construed as limiting the scope of the invention as other equivalent embodiments will be apparent in view of the present disclosure and appended claims.

### Preparation of Copolymers

PEA-X25 is a random copolymer within the scope of PEA polymers as described herein and has the structure:

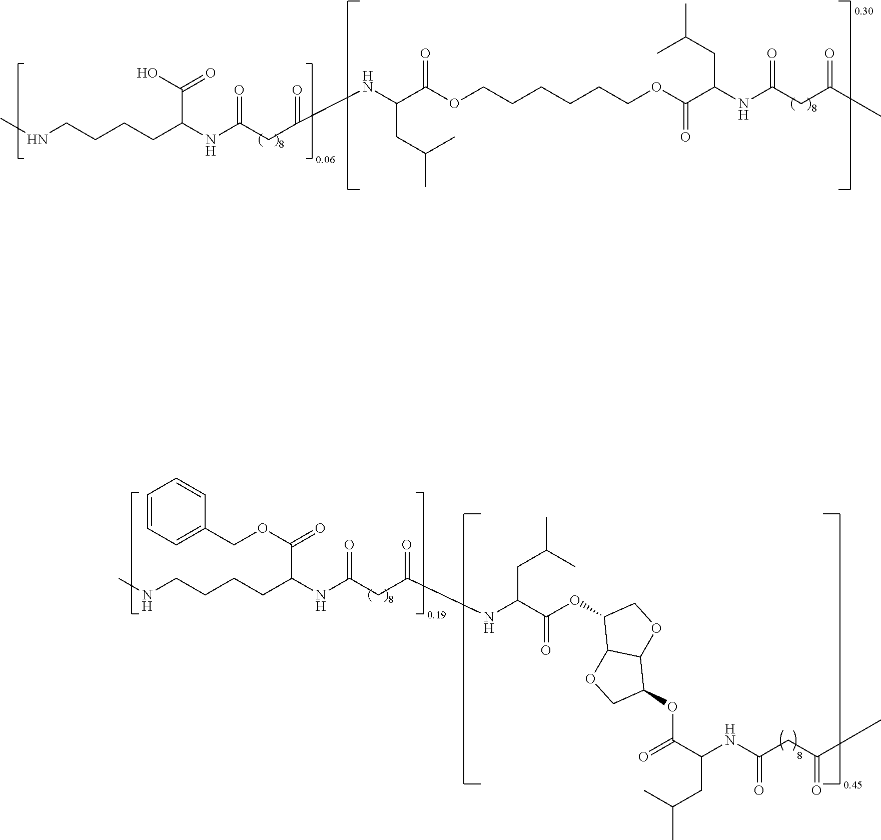

This PEA polymer therefore has monomer residues with the structures as described generally herein, in which m is 0.30, p is 0.45, q is 0.19, and x is 0.06; R¹ is octylene [(C₈)alkylene]; R³ and R⁴ are isobutyl; R⁵ is hexylene [(C₆)alkylene];
R⁶ is

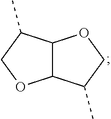

R⁷ is benzyl [Ph-(C₁)alkylene, or phenylmethyl]; and R⁸ is butylene [(C₄)alkylene]. This PEA copolymer was prepared according to the following description.

Triethylamine (31 ml, 0.222 mole) and DMSO (54 ml, 0.76 mole) were added to a mixture of di-N-hydroxysuccinimide ester of sebacic acid (Di-NHS-sebacic acid) (39.336 g, 0.099 mole), L-leucine-(DAS)-2TosOH (32.876 g, 0.045 mole), L-leucine(6)-2TosOH (21.062 g, 0.030 mole), L-lysine.2HCl (1.396 g, 0.006 mole) and L-lysine (Bz)-2TosOH (4.235 g, 0.018 mole) in a nitrogen flushed 500 ml round bottomed flask equipped with an overhead stirrer at room temperature. The subsequent mixture was heated to 60° C. to allow the reaction to proceed, with monitoring by GPC analysis in THF. After 36 hours, a stable molecular weight was obtained. The reaction mixture was diluted with 250 ml DMSO and was allowed to cool to room temperature. At room temperature, acetic anhydride (1.89 ml, 0.0199 mole) was added to acylate the amino functional end groups of the polymer. Next, the mixture was stirred at room temperature for 24 hours.

The obtained crude polymer mixture was precipitated in water at a 10:1 ratio (water: reaction mixture). The polymer was collected and dissolved in ethanol (500 ml, 8.57 mole) and then precipitated a second time. The polymer was again dissolved in ethanol (500 ml, 8.57 mole) and precipitated in ethylacetate (5000 ml, 50.91 mole) by dropwise addition to a stirring solution. The precipitated polymer was washed with ethylacetate (100 ml, 1.00 mole), the supernatant was removed, and the precipitate was washed again with ethylacetate (100 ml, 1.00 mole). After the removal of the supernatant, the precipitate was dried and dissolved in ethanol (500 ml, 8.57 mole), and filtered over a 0.2 μm PTFE membrane filter. The filtered polymer solution was dried under reduced pressure at 65° C. From this synthesis procedure, a typical yield is 75%, and the number average molecular weight (Mn) is normally in the range of 40-70 kDa, measured via gel permeation chromatography (GPC) using tetrahydrofuran (THF) as the mobile phase on dried samples, and determined relative to polystyrene standards.

PEA III AcBz is a random copolymer within the scope of PEA polymers as described herein and having the structure:

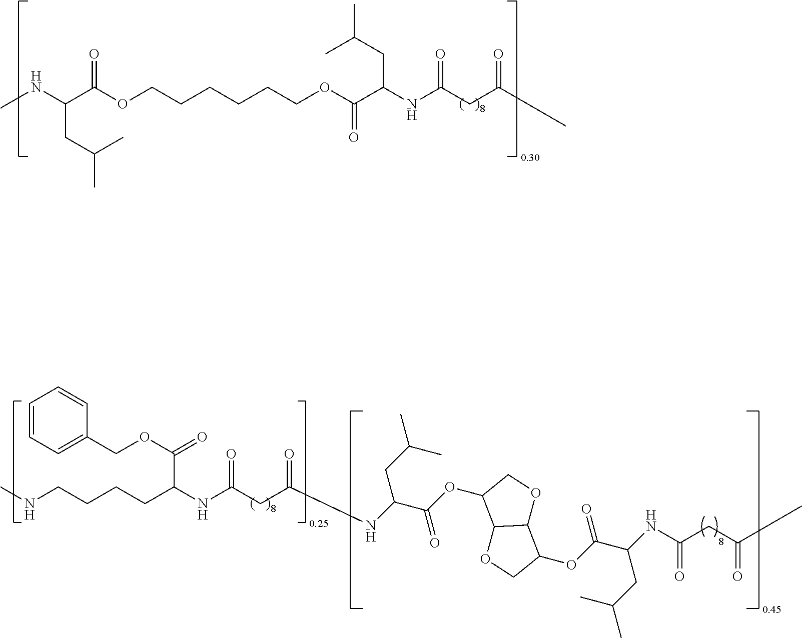

This PEA polymer therefore has monomer residues with the structures as shown above, in which m is 0.3, p is 0.45, q is 0.25, and x is 0; R¹ is octylene [(C₈)alkylene]; R³ and R⁴ are isobutyl; R⁵ is hexylene [(C₆)alkylene];
R⁶ is

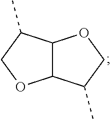

R⁷ is benzyl [Ph-(C₁)alkylene, or phenylmethyl]; and R⁸ is butylene [(C₄)alkylene]. This PEA copolymer was prepared according to the following description.

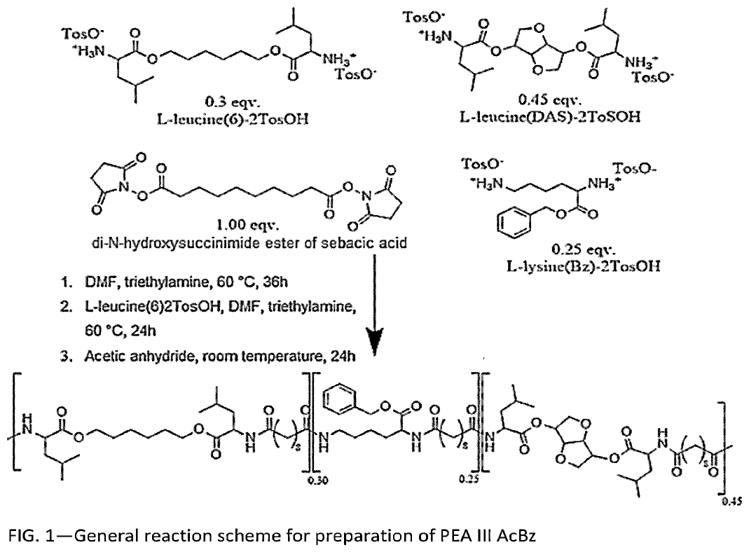

Triethylamine (30.9 ml, 0.222 mole, 2.2 eq) and N,N-dimethylformamide (53.07 ml, 0.689 mole) were added to a mixture of di-N-hydroxysuccinimide ester of sebacic acid (Di-NHS-sebacic acid) (39.940 g, 0.1008 mole, 1.0 eq), L-leucine(6)-2TosOH (20.823 g, 0.0302 mole, 0.30 eq), L-leucine-(DAS)-2TosOH (32.503 g, 0.0453 mole, 0.45 eq) and L-lysine(Bz)-2TosOH (14.628 g, 0.0252 mole, 0.25 eq) in a nitrogen flushed 500 ml round bottomed flask equipped with an overhead stirrer at room temperature. The subsequent mixture was heated to 60° C. to allow the reaction to proceed, with monitoring by GPC analysis in THF. After 36 hours, a stable molecular weight was obtained. Subsequently, a portion of L-leucine(6)-2TosOH (4.338 g, 0.0063 mole) along with triethylamine (1.76 ml, 0.0126 mole) and N,N-dimethylformamide (4.54 ml, 0.0590 mole) were added to terminate the polymerization reaction. The mixture was heated additionally for 24 hours, after which the viscous solution was further diluted with N,N-dimethylformamide (407.85 g, 5.301 mole) and allowed to cool to room temperature. At room temperature, acetic anhydride (1.89 ml, 0.0199 mole) was added to acylate the amino functional end groups of the polymer. The mixture was stirred at room temperature for 24 hours. The general reaction scheme is shown in FIG. 1.

The obtained crude polymer mixture was precipitated in water at a 10:1 ratio (water: reaction mixture). The polymer was collected and dissolved in ethanol (500 ml, 8.57 mole) and then precipitated a second time. The polymer was again dissolved in ethanol (500 ml, 8.57 mole) and precipitated in ethylacetate (5000 ml, 50.91 mole) by dropwise addition to a stirring solution. The precipitated polymer was washed with ethylacetate (100 ml, 1.00 mole), the ethylacetate removed, and then the polymer was washed in ethylacetate again (100 ml, 1.00 mole). The polymer was then dried and dissolved in ethanol (500 ml, 8.57 mole) and filtered over a 0.2 μm PTFE membrane filter. The filtered polymer solution was dried under reduced pressure at 65° C. The yield was 75%, and the number average molecular weight (Mn) was 43.3 kDa, measured via gel permeation chromatography (GPC) using tetrahydrofuran (THF) as the mobile phase on dried samples, and determined relative to polystyrene standards.

### Mechanical Properties of PEA and Determination of Wet Tg

In preparation for mechanical measurements, samples were compression molded at 120° C. under N₂ atmosphere into disc shaped samples with a diameter of 25 mm and a thickness of ˜0.5 mm. Several smaller samples with 4 mm diameter were punched from the larger discs.

### Shear Storage Modulus in Dry Conditions

Prior to measurement, sample discs were dried for 16-40 hours at 65° C. at 150 mbar (N₂ atmosphere). The measurements were performed on an ARES2-rheometer using 4 mm parallel plates. A temperature ramp test was performed from 90 to 0° C. (cooling at 5° C./min) at an angular frequency of 1 Hz (6.28 rad/s) and a variable strain (autostrain control enabled) on the sample (FN˜30 grams). This constant compressive force was necessary to prevent a loss of contact between the sample and the parallel plates. Shear storage modulus, loss modulus, and phase angle were recorded as a function of temperature. The value of the storage modulus (G′) at a temperature of 37° C. was reported.

### Determination of Tg in Wet Conditions

Prior to measurement, the sample discs were saturated in a PBS buffer solution (Dulbecco w/o Ca²⁺ w/o Mg²⁺) at room temperature for >100 hours. The measurements were performed on an ARES2-rheometer using disposable 15 mm parallel plate geometries with a solvent trap filled with PBS buffer. This created a saturated atmosphere to prevent evaporation of moisture from the sample. In this geometry, the sample was not in direct contact with PBS. For some measurements, an alternative geometry consisting of a 4 mm upper plate was used, in which the sample was immersed in PBS buffer on the bottom plate. A temperature ramp test was performed from 45 to 0° C. (cooling @ 5° C./min) at an angular frequency of 1 Hz (6.28 rad/s) and a variable strain (autostrain control enabled) with an initial value of 0.1%. The gap was controlled manually to ensure a constant axial force (compression) on the sample (FN˜30 grams). This constant compressive force was necessary to prevent a loss of contact between the sample and the parallel plates. Shear storage modulus, loss modulus, and phase angle were recorded as a function of temperature. Glass transition temperature (Tg) was reported as the temperature at which the loss modulus (G″) reached a maximum value. A summary of results, in terms of (i) shear storage modulus (G′) at a frequency of 1 Hz in the dry state at 37° C. and (ii) glass transition temperature in the hydrated state (wet Tg) for various PEA samples is provided in Table 1 below:

TABLE 1

| Sample | G′ at 37° C., dry (Pa) | Tg, wet (° C.) |
|---|---|---|
| PEA-X25 | 3.7 × 10⁸ (1) | 21.4 (2) |
| PEA-X50 | 3.4 × 10⁸ (1) | 25.8 |
| PEA-X75 | 3.5 × 10⁸ | 27.4 |
| PEA (p:q = 1:1) | 4.0 × 10⁸ | 23.9 |
| PEA (p:q = 75:25) | 3.9 × 10⁸ | 31.5 |
| PEA (p:q = 85:15) | >1 × 10⁸ | 38.8 |
| PEA-III AcBz (batch 1) | 2.72 × 10⁸ | 21.1 |
| PEA-III AcBz (batch 2) | 2.47 × 10⁸ | 23.6 |

A description of the random copolymers, PEA-X25 and PEA-III AcBz, and their preparation methods is provided above. PEA-X50 and PEA-X75 are variations of PEA-X25, in which q:x=1 and q:x=1:3, respectively. The other random copolymers referenced in Table 1 above have variations in terms of their p:q ratios as indicated. Results of G′ reported with (1) indicated that the value was obtained at the temperature at which the measurement stopped (between 40 and 50° C., already in the glassy plateau/below Tg). The value was almost constant in this range. The result of Tg reported with (2) indicated that the sample was measured on an alternative geometry in contact with the PBS buffer. Given this information, those skilled in the art can appreciate various parameters affecting shear storage modulus and wet Tg.

### Water Uptake of PEA

Water uptake measurements were performed on free floating films in phosphate buffered saline (PBS) at 37° C. Buffers were exchanged every two weeks to prevent buildup of degradation products. Sacrificial type sampling was done in triplicate at the required times. Samples were blotted on dust-free tissue in order to remove adhering buffer, and then weighed on a microbalance. The water uptake was reported as a percentage of the original sample mass. A summary of results, in terms of water uptake over time, starting from a dry state, for various PEA samples is provided in Table 2 below.

TABLE 2

| time | PEA-III | Water uptake (%) | | | |
|---|---|---|---|---|---|
| (days) | AcBz | PEA-X25 | PEA-X50 | PEA-X75 | PEA-X100 |
| 1 | 2.9 | 3.5 | 3.5 | 24.0 | 9.5 |
| 2 | 3.3 | 4.0 | 5.8 | 43.2 | 14.1 |
| 4 | 3.4 | 4.1 | 8.3 | 55.7 | 19.2 |
| 7 | 3.8 | 4.9 | 11.1 | 75.5 | 27.9 |
| 15 | 2.9 | 4.9 | 15.6 | 159.6 | |
| 24 | 2.9 | 5.9 | 20.9 | 265.2 | |
| 37 | 2.9 | 7.4 | 28.7 | 229.3 | |
| 51 | 2.5 | 8.3 | 31.4 | 201.2 | |

A description of the random copolymers, PEA-X25 and PEA-III AcBz, and their preparation methods is provided above. PEA-X50 and PEA-X75 are variations of PEA-X25, in which q:x=1 and q:x=1:3. PEA-X100 is another variation, in which q=0 and x=0.25. From the above results, PEA-III AcBz and PEA-X25 showed very little change in water uptake over two weeks, the approximate maximum lifetime of glucose sensors, such that these copolymers provided desirable properties from the standpoint of stability under physiological conditions. PEA-X50 and PEA-X75 exhibited less constant water uptake performance, but could be appropriate for use according to some embodiments described herein. Given this information, those skilled in the art can appreciate various parameters affecting water uptake stability.

### Film Preparation for Transport Property Measurements

With respect to PEA film preparation for oxygen permeability measurements, PEAs were dissolved in THF to obtain a 10 wt-% solution. Approximately 6 grams of the solution was added to a glass petri dish with a diameter of 7 cm. After evaporation of the THF, another 2 grams of the PEA solution was added. After further evaporation, a film thickness of approximately 225 microns was obtained (average of 10 random spots). For complete removal of THE, the films were stored in an oven at 50° C./900 mbar for 16-24 hrs. With respect to PEA film preparation for glucose permeability measurements, PEAs were dissolved in THF to obtain a 15 wt-% solution. A film was drawn on a PE substrate with an applicator with a gap of 1000 microns. The solvent was allowed to evaporate overnight. A second film with the PEA solution was drawn on top of the previous formed film, and the solvent was likewise allowed to evaporate overnight. A film thickness of approximately 100 microns was obtained. For complete removal of THF, the films were stored in an oven at 50° C./900 mbar for 16-24 hrs.

### Glucose Permeability

Films prepared as described above were cut in the shape of a circle (diameter ˜6 cm) to remove any curled edges or air bubbles. The films were hydrated in distilled water prior to testing. For each film, the thickness was measured 10 times using a thickness gauge. The average of the 10 measurements was used as the film thickness. The film was then mounted between two silicone gaskets with vacuum grease. The inner diameter of the gaskets was ˜5 cm. The gaskets were then mounted on a Franz cell apparatus with the receptor cavity filled with deionized water. The receptor vessel was equipped with sampling ports, in which samples could be removed over time. A top was placed on the receptor vessel that sealed the film onto the Franz cell and created the donor vessel. A concentrated glucose solution (2000 mg/dl) was inserted into the donor vessel. The Franz cell was then closed with a lid to prevent evaporation and placed in a 37° C. shaker oven having a stage set to shake at 150 rpm. Samples of 100 microliters each were taken over a period of 6 hours. Deionized water was used to replace the liquid in the donor vessel and maintain sink conditions.

The liquid samples were then analyzed for glucose content using a glucose colorimetric enzyme assay and a UV plate reader. The amount of glucose in each sample was determined using a calibration curve. The slope of mg glucose/cm² over time was determined and converted into a flux. This flux value was then utilized to calculate the permeability of the glucose of each film. All films were analyzed in at least duplicate to ensure no leaks were present. The glucose permeability was computed as the average of two samples, according to Fick's first law:

$$J = - {D}_{G} \frac{dc}{dx} ,$$

where J is the glucose flux, D_{G} is the diffusion coefficient of glucose in the sample, c is the concentration of glucose in the sample, and x is the spatial coordinate across the sample thickness. The glucose concentration in the sample at the donor cell interface is denoted c_{S,D} and is equal to

c_{S,D}−∝_{G}c_{D},

where c_{D} is the concentration of glucose in the donor cell, and ∝_{G} is the partition coefficient of glucose between the sample and water, following the naming convention of Leypoldt 1984 (DOI: 10.1021/ac00278a063). This partition coefficient as defined is unitless. Similarly, the glucose concentration at the acceptor cell interface is denoted c_{S,A} and is equal to

c_{S,A}=∝_{C}c_{A},

where c_{A} is the concentration of glucose in the acceptor cell. After an initial time lag, a quasi-steady-state is reached where the concentration gradient across the sample thickness is linear and the flux expression can be approximated by

$$J \approx {D}_{G} \frac{( {c}_{S , D} - {c}_{S , A} )}{h} \approx \frac{{D}_{G} {∝}_{G} {c}_{D}}{h} ,$$

since the concentration in the acceptor cell is always negligible, relative to the concentration of the donor cell in this experiment. The glucose permeability of the sample is then computed according to

$${P}_{G} \_ {D}_{G} {∝}_{G} \_ \frac{Jh}{{c}_{D}} .$$

Note that as the partition coefficient is unitless, the permeability has identical units as diffusivity.

### Oxygen Permeability

Films prepared as described above were conditioned in a 23° C./50% relative humidity (RH) lab environment prior to testing. Film thickness was measured in triplicate with a Magna-Mike 8600 and averaged. Because the samples absorb water and swell somewhat during the measurement performed at 90% RH, if they are not constrained they can bulge and thereby change the test area. To prevent this, the substrates were sandwiched between a flat plastic film of known oxygen transmission resistance (on the acceptor cell side) and an aluminum mask with a circular hole, with epoxy glue being used to seal the borders on both sides to constrain the sample and prevent leaks. This was mounted against a metallic grid (on the donor cell side). The test area was the size of the sample exposed in the circular hole of the mask and was measured for each sample. The film was therefore physically constrained on both sides and did not bulge when the sample rehydrated, thus keeping the test area constant.

The assembly was mounted onto a Mocon OxTran 2/21 SH2-module system and the oxygen transmission rate was measured, utilizing humidified air (20.9% O₂) as the test gas and humidified nitrogen as the carrier gas. The humidity on both sides was 90% RH. Once enough time had elapsed for the sample to equilibrate to the higher humidity and the oxygen transmission rate had stabilized, the oxygen transmission resistance of the stacked sample was calculated and the known resistance of the plastic substrate was subtracted to obtain the resistance of the PEA film. Film thickness was measured again after the experiment. For these samples, it was observed not to change significantly. The oxygen transmission rate was rescaled to a 100% oxygen basis for reporting purposes and was the average of two samples.

In this gas-to-gas method of measuring oxygen permeability, it was not possible to fully hydrate the test specimens (100% RH) as this would have led to condensation of water on the sample that would interfere with the results. The results therefore represented the oxygen permeability at slightly less than full hydration. As water acts as a plasticizer to this class of PEA polymers, increasing chain mobility as evidenced by the lowering of the glass transition temperature (Tg), the addition of more water (full hydration) would presumably result in even more chain mobility. As oxygen permeability is helped by polymer chain mobility, the values reported here at 90% RH were presumed to underestimate oxygen permeability at full hydration on an implanted sensor coating.

As in the case of glucose permeability, oxygen permeability was also calculated on the basis of Fick's first law according to

$$J = - {D}_{O} \frac{dc}{dx} ,$$

where D_{O} is the diffusion coefficient of oxygen in the sample and c now refers to oxygen concentration. Once again at quasi-steady-state the concentration gradient in the sample is linear and the expression can be approximated as

$$J \approx {D}_{O} \frac{( {c}_{S , D} - {c}_{S , A} )}{h} \approx \frac{{D}_{O} {c}_{S , D}}{h} .$$

The oxygen concentration in the sample at the donor cell interface is expressed according to Henry's law

c_{S,D}=k_{S}p_{O}_{₂},

where k_{S} is the Henry's law constant for oxygen in the sample, and p_{O}_{₂} is the partial pressure of oxygen in the donor cell. As the reported flux value has been rescaled to a 100% oxygen basis, p_{O}_{₂} is equal to atmospheric pressure or 1.013 bar. The oxygen permeability of the sample is therefore

$${D}_{O} {k}_{S} = \frac{Jh}{{p}_{{O}_{2}}} .$$

As oxygen is a gas, oxygen permeability is typically defined in this way. However, in a glucose limiting membrane, the membrane would be in contact with water, therefore in order to compare with the glucose permeability to calculate a permeability ratio it is necessary to express the oxygen permeability in a different form. For this we define ∝_{O} as the unitless partition coefficient of oxygen between the sample and water. Since Henry's law constants are partition coefficients between gas and liquid, ∝_{O} can be related to them according to

$${α}_{O} = \frac{{k}_{S}}{{k}_{W}} ,$$

where k_{W} is the Henry's law constant for oxygen in water. NIST reported values for oxygen in water yield a value for k_{W} of 0.00107 mol/kg·bar at 37° C. With some unit conversion a value for the oxygen permeability of the sample was computed in the same units as the glucose permeability (cm²/s):

$${D}_{O} {α}_{O} = \frac{Jh}{{p}_{{O}_{2}} {k}_{W}} .$$

The oxygen to glucose permeability ratio was then calculated as

$$\begin{matrix}{P}_{O} \\ {P}_{G}\end{matrix} = \begin{matrix}{D}_{O} {α}_{O} \\ {D}_{G} {α}_{G}\end{matrix} .$$

A summary of results, in terms of glucose permeability, oxygen permeability, and the oxygen: glucose permeability ratio, for various PEA samples is provided in Table 3 below.

TABLE 3

| | Glucose | Oxygen | |
|---|---|---|---|
| | permeability | permeability | O/G permeability |
| Sample | (cm²/s) | (cm²/s) | ratio |
| PEA-X25 | 5.6 × 10⁻¹¹ | 3.0 × 10⁻⁷ | 5.3 × 10³ |
| PEA-X50 | 1.6 × 10⁻¹¹ | 3.0 × 10⁻⁷ | 2.0 × 10⁴ |
| PEA-IIIAcBz | n.d. | 3.6 × 10⁻⁷ | n.d. |

A description of the random copolymers, PEA-X25 and PEA-III AcBz, and their preparation methods is described above. PEA-X50 is a variation of PEA-X25, in which q:x=1. Water has a glucose permeability of approximately 10⁻⁵ cm²/s at 37° C. From the above results, the glucose permeability values of the PEA samples were much lower, indicating that PEA polymers would significantly limit the transport of glucose to the sensor. They are, however, non-zero, indicating that they would allow some glucose through and that the coating could be engineered to be of the appropriate thickness to bring the sensor response in the desired range. Surprisingly, despite the oxygen permeability being measured at slightly less than full hydration (and therefore being an underestimate), the oxygen permeability was several orders of magnitude larger than the glucose permeability. This results in the oxygen to glucose permeability ratio being significantly higher than 440, a target value to be achieved in some embodiments.

### Preparation of Coatings—Window on a Mouse Model

For in vivo experimentation with PEA-X25 and PLGA 85:15 (conventional biodegradable polymer), coatings for a window on a mouse model were prepared by pipetting 100 μl of the solutions (5.9% w/w in THF) onto glass cover slides with a diameter of 12 mm and dried under vacuum at room temperature. Glasses were cleaned before use by rinsing with ethanol, wiping with spec wipe tissue, and drying in air.

### In Vivo Studies and Collagen Mapping—Window on a Mouse Model

A total of forty mice were kept two weeks after arrival for acclimatization and were 8-12 weeks old at the time of implantation. Throughout the experiments, the animals were kept on an alfalfa free diet. Access to food and water was ad libitum. Macrophage Fas induced apoptosis mice (MaFIA, 12 mice) (Jackson lab, Bar Harbor, ME, USA), strain C57BL/6-Tg(Csf1r-EGFPNGFR/FKBP1A/TNFRSF6)2Bck/J) were used in the 2-photon-microscopy experiments for imaging the production of eGFP-tagged CD115 expressing cells in these mice. CD115 is present in monocytes/macrophages, peritoneal exudate cells, plasmacytoid and conventional dendritic cells, and osteoclasts. Apoptosis of macrophages was not induced in the animals. In addition, second harmonic generation imaging was applied to visualize and quantify collagen deposition.

The implantation process occurred as described in Ritsma et al. (NAT. PROTOC. 2013 (8): 583-594). Briefly, anesthesia was administered in an induction cabinet using 4% isoflurane in oxygen before being maintained at 2% isoflurane. The right flanks of the mice were shaved and disinfected with 70% ethanol. An approximately 17 mm long lateral incision was made in the dorsolateral portion of the skin. Next, the underlying fascia was separated from the skin by blunt dissection. A purse string suture was made around the edge of the incision using a polypropylene suture (Prolene® 5-0, Ethicon, Somerville, NJ, USA). The PEA- or PLGA-coated windows were then inserted in the incision and the sutures drawn tight and fixed. Buprenorphine 0.05 mg/kg was administered for pain relief and mice were allowed to recover in a prewarmed box till the end of the day. Each animal was then transferred to a prewarmed 37° C. closed microscopy stage and anesthesia was maintained with 2% isoflurane delivered by a tube in which the snout of the mice was placed. The animals were positioned for 2-photon microscopy, second harmonic generation imaging. At the end of the experiments, the mice were sacrificed by cervical dislocation after being anaesthetized with isoflurane. The imaging windows were then removed with the associated tissue that was attached to the implant.

### Quantification of Collagen Deposition Around the Coated Windows In Vivo

Collagen around PEA and PLGA based coatings was determined with intravital imaging and machine learning. In particular, 2 photon confocal laser scanning microscopy was used to study deposition of collagen fibers (based on second harmonic generation (SHG) imaging). Briefly, SHG is a process that occurs when two photons are combined in an optically nonlinear medium, lacking in centro-symmetry (such as collagen), creating a SHG photon with a wavelength exactly half of the excitation wavelength (or twice the frequency, ω). Collagen fibers have a very suitable structure for generating SHG signal. Fibrillary collagen is highly anisotropic and the SHG signal generated is coherently amplified because of the tight alignment of repeating structures within the collagen triple helix and within fibrils. Thus, signal intensity correlates with collagen content.

The development of host responses were imaged using two photon-confocal microscopy. In each imaging session, anesthesia was induced with 4% isoflurane in oxygen in an induction box. The animal was then transferred to a prewarmed 37° C. closed microscopy stage and anesthesia was maintained with ˜2% isoflurane delivered by a tube in which the snout of the mice was placed. The animal was positioned in such a way that the window faced upwards and could be fixed using a custom-built clamp on a fixed crane. Imaging was performed using a Zeiss LSM 7MP microscope (Zeiss, Oberkochen, Germany) with a Chameleon Vision compact OPO two-photon laser. The use of second harmonics generation for imaging of biological structures possessing large hyper polarizabilities, in this case collagen fibers, was applied using 840 nm excitation in combination with a 380-430 nm emission filter. Images were acquired using ZEN imaging software (Zeiss). The Zeiss “czi” files were converted into HDF5 format files using the python packages “czifile” version 2019.7.2 and “h5py” version 2.10.0. The original resolution and 8-bit depth were lossless preserved in the converting process. For the collagen quantification, only the white channel of the data was used. The sizes of the images were 512×512 pixels, in the depth direction the size of the files varied, but all files contained at least 100 μm=100 pixels. The voxel dimensions were 1.19×1.19×1 μm.

### White Value

For each 2D slice, the average value of the white channel was calculated, resulting in a value between 0 and 255 as a function of depth, where a depth of zero was close to the window/coating and a depth of 100 μm was further into the mouse.

### White Fraction

For each 2D slice, the fraction of pixels that have a white intensity over 128 was calculated, resulting in a value between 0 and 1 as a function of depth where a depth of zero was close to the window/coating and a depth of 100 μm was further into the mouse. A value of 0 represented a slice with only black and dark gray pixels, and a value of 1 represented a slice with only light gray and white pixels. First, all values over 5 micrometers were averaged, and thereafter all data from 2 different spots in 3 different mice were taken together for both PEA and PLGA coatings and these values were clustered into three blocks (0-30 μm, 30-60 μm and 60-100 μm). These 6 datasets were entered into a non-parametric, one-way ANOVA (Kruskal Wallace test) to calculate significance between each of the datasets.

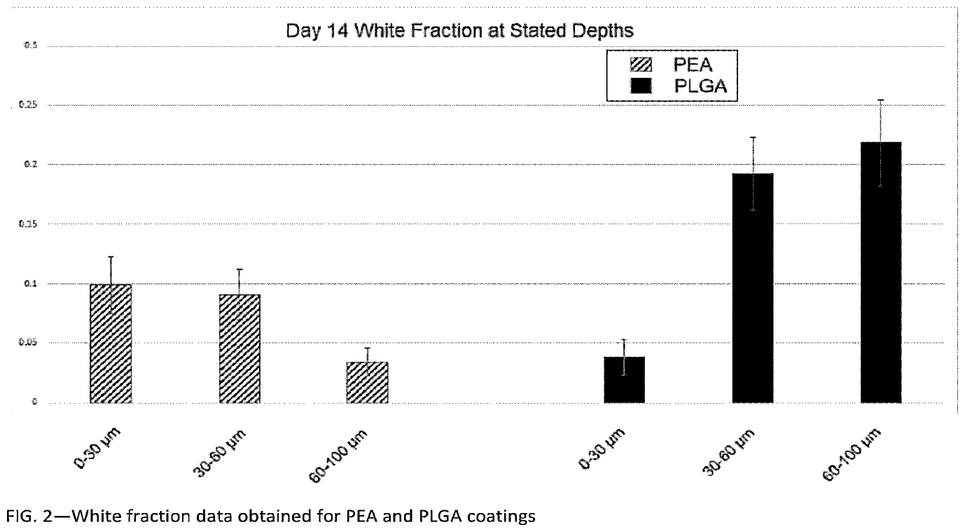

FIG. 2 provides a bar graph of the white fraction data obtained with the PEA and PLGA coatings, following a 14-day experimental period in which the PEA-or PLGA-coated windows were implanted, as described above. In particular, this graph shows the white fraction at each of the three depth level range clusters noted above. Essentially, the white fraction provides a measure of collagen fibrils, with greater amounts of these fibrils translating to more collagen overall. Considering that glucose must pass through all collagen depth levels to reach a sensor, the total amount of collagen, represented by the combined amounts given by each of the three bars, is the relevant basis for comparison. As is apparent from FIG. 2, the PEA polymer led to significantly reduced collagen deposition compared to the PLGA polymer, thereby demonstrating advantageous properties of PEA polymers as described herein, for use in membranes and coatings for implantable devices such as those used in CGM.

### Preparation of Coated Filaments—Porcine Punch Biopsy Study

7 cm stainless steel filaments were dip coated with 10% w/w ethanol solutions of PEA-X25 using the RDX coating system. All samples were dip coated twice to give a coating thickness ranging from 9-12 micron. Sensor probe filaments were cut from an Abbott FreeStyle Libre FGM and remained uncoated as a control. The PEA-X25 coated filaments and the commercial filaments were compared for their tissue response in a punch biopsy study detailed below.

### In Vivo Study—Porcine Punch Biopsy Study

A Yucatan non-diabetic swine model was used to evaluate and compare the tissue reaction between the coated filaments and the commercial filaments. Studies were terminal. Thus, a maximum biopsy on each of the three available swine was conducted. Samples were evenly distributed between the three swine at a total of 130 samples. As this was a terminal study the number of implants per animals was not restricted.

Preimplantation preparation is as follows. Swine is fasted overnight and sedated the next morning prior to implantation. Skin over the lateral chest and abdomen is shaved, lightly cleaned with soap followed by alcohol wipes. Anesthetic depth is monitored by the veterinarian staff and not the surgeon performing the implantation procedure.

Implantation is performed as follows. An 18-gauge needle is used to carefully pick up the material and allow it to slide into the needle shaft. With the material inside the needle shaft with or without the addition of injectable saline, the material was injected subcutaneously into the swine skin. At times, material retracted or did not inject into the subcutaneous tissue site. On occasion, the material was still found inside the needle shaft. If this was the case, the material was manually pushed into the tissue. The implantation site was marked with a permanent marker and taped over.

Tissue reactions are evaluated longitudinally over up to 14 consecutive days at sites of material location via punch biopsy. Specifically, coated filament and commercial filament effects are evaluated at either 1 or 2 days, 7 and 14 days of implantation. Material for histopathological evaluation was obtained through punch biopsies, processed for H&E, fibrosis (trichrome) and immunohistochemistry (IHC) for endothelial cell markers (e.g. blood vessels). Excised tissue samples are examined by IHC and histopathology in order to compare the cellular response, time course, and response severity. Pathology assessment includes assessment of fibrin capsule formation at the material implantation site. Fibrosis in porcine are detected using Masson Trichrome dye system. Using this system, connective tissue stains blue, nuclei stains dark red/purple, and cytoplasm stains red/pink. Blood vessels in tissue sections are detected using antibodies to CD31 (Abcam) and standard IHC. Small and intermediate vessels stain brown. Matched non-immune IgG did not experience any specific or non-specific CD31 staining for all treatment conditions (data not shown). All photomicrographs were taken at 4×, 10× or 20× as designated.

Masson Trichrome staining revealed that there was significantly less (30-70%) collagen deposition with the coated filaments compared with commercial filaments at 7 and 10 days. Collagen deposition is known to interfere with sensor performance as indicated earlier. A clear benefit in terms of tissue response with the PEA materials was demonstrated.

Overall, aspects of the invention relate to the discovery that PEA polymers described herein can possess a number of important advantages for providing longer performing glucose sensors and/or for simplifying their design. For example, in vivo modeling has confirmed that these polymers exhibit both decreased collagen deposition and increased neovascularization (angiogenesis). Additional testing has demonstrated favorable oxygen and glucose transport capabilities, as well as other relevant properties. Those skilled in the art having knowledge of the present disclosure, will recognize that various changes can be made to these polymers and their applications, to attain these and other advantages, without departing from the scope of the present disclosure. As such, it should be understood that the features of the disclosure are susceptible to modifications and/or substitutions without departing from the scope of the invention. The specific embodiments illustrated and described herein are for illustrative purposes only, and not limiting of the invention as set forth in the appended claims.

### EXEMPLARY DESCRIPTION OF CERTAIN EMBODIMENTS

1. A glucose limiting membrane for use in continuous glucose monitoring (CGM), said glucose limiting membrane comprising a polyesteramide (PEA), wherein the PEA has a structure including n units of:
m molar equivalents of a first monomer residue having the formula

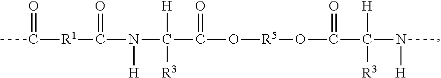

p molar equivalents of a second monomer residue having the formula

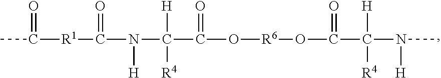

q molar equivalents of a third monomer residue having the formula

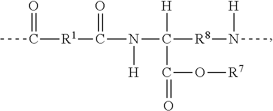

and
x molar equivalents of a fourth monomer residue having the formula

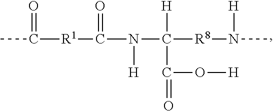

wherein m+p+q+x=1;
m is from 0 to 0.8, p is from 0 to 0.95, m+p is from is from 0.5 to 1, q is from 0 to 0.35, and x is from 0 to 0.25;
n is from 5 to 300;
R¹ is (C₂-C₂₀)alkylene;
R³ and R⁴ are selected from the group consisting of hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃⁺, —(CH₂)₃NHC(═NH₂⁺)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph—CH₂—, CH═C—CH₂—, (CH₃)₂CH—, Ph—NH—,

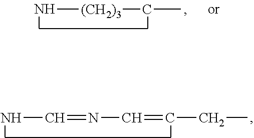

wherein R³ and R⁴ are the same or different;
R⁵ is (C₂-C₂₀)alkylene, optionally having one or more alkylene carbon atoms (—C—) replaced by an oxygen atom (—O—), and/or optionally having 5 or 6 consecutive alkylene carbon atoms together form a respective 5- or 6-membered carbocyclic ring;
R⁶ is

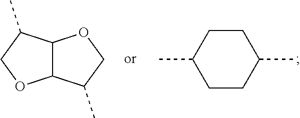

R⁷ is (C₆-C₁₀)aryl(C₁-C₆)alkylene; and
R⁸ is (C₃-C₈)alkylene.
2. The glucose limiting membrane of exemplary embodiment 1, wherein, in the structure of the PEA, p is from 0 to 0.8 and m+p is from is from 0.5 to 0.9.
3. The glucose limiting membrane of exemplary embodiment 1 or 2, wherein, in the structure of the PEA, q is from 0.05 to 0.25.
4. The glucose limiting membrane of any one of exemplary embodiments 1-3, wherein, in the structure of the PEA, x is from 0.05 to 0.25.
5. The glucose limiting membrane of any one of exemplary embodiments 1-4, wherein, in the structure of the PEA, q:x is from 9:1 to 1:4.
6. The glucose limiting membrane of any one of exemplary embodiments 1-5, wherein, in the structure of the PEA, q:x is from 4:1 to 1:4.
7. The glucose limiting membrane of exemplary embodiment 1 or 2, wherein, in the structure of the PEA, q is 0.
8. The glucose limiting membrane of exemplary embodiment 1 or 2, wherein, in the structure of the PEA, x is 0.
9. The glucose limiting membrane of exemplary embodiment 1 or 3-8, wherein, in the structure of the PEA, m is 0.
10. The glucose limiting membrane of exemplary embodiment 1, wherein the structure of the PEA is further characterized by one or more of the following:
m is from 0.1 to 0.5, p is from 0.1 to 0.75, m+p is from is from 0.5 to 0.8, q is from 0.1 to 0.35, and x is from 0 to 0.1;
R¹ is (C₂-C₁₀)alkylene;
R³ and R⁴ are (C₁-C₆)alkyl;
R⁵ is (C₂-C₁₀)alkylene;
R⁶ is

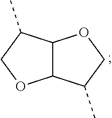

R⁷ is Ph-(C₁-C₆)alkylene; and
R⁸ is (C₃-C₆)alkylene.
11. The glucose limiting membrane of exemplary embodiment 10, wherein the structure of the PEA is further characterized by one or more of the following:
m is 0.3, p is 0.45, q is 0.25, and x is 0;
R¹ is octylene;
R³ and R⁴ are isobutyl;
R⁵ is hexylene;
R⁶ is

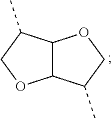

R⁷ is benzyl; and
R⁸ is butylene.
12. The glucose limiting membrane of any one of exemplary embodiments 1 to 11, having an oxygen to glucose permeability ratio of at least about 440.
13. The glucose limiting membrane of any one of exemplary embodiments 1 to 12, having a glucose permeability of at least about 10⁻¹⁵ cm²/s.
14. The glucose limiting membrane of exemplary embodiment 13, wherein the glucose permeability is at least about 10⁻¹² cm²/s.
15. The glucose limiting membrane of any one of exemplary embodiments 1 to 14, wherein the PEA has a glass transition temperature in the hydrated state of less than about 37° C.
16. The glucose limiting membrane of any one of exemplary embodiments 1 to 15, wherein the PEA is amorphous.
17. The glucose limiting membrane of any one of exemplary embodiments 1 to 16, wherein the PEA has a water uptake of less than about 30 wt-% after two weeks in phosphate buffered saline (PBS) at 37° C.
18. The glucose limiting membrane of any one of exemplary embodiments 1 to 17, wherein the PEA has a water uptake that remains within about +/−20%, over a period from 4 days to two weeks in phosphate buffered saline (PBS) at 37° C.
19. The glucose limiting membrane of any one of exemplary embodiments 1 to 18, wherein the PEA in the dry state at 37° C. has a shear storage modulus G′ at a frequency of 1 Hz of at least about 0.1 MPa.
20. The glucose limiting membrane of any one of exemplary embodiments 1 to 19, wherein the membrane does not include an anti-inflammatory agent.
21. The glucose limiting membrane of any one of exemplary embodiments 1 to 20, further comprising an additional polymer.
22. The glucose limiting membrane of exemplary embodiment 21, wherein the additional polymer underlies an external surface layer of the PEA, said external surface layer being configured for direct contact with interstitial fluid of the patient.
23. An implantable glucose sensor comprising a coating comprising a polyesteramide (PEA), wherein the PEA has a structure including n units of:
m molar equivalents of a first monomer residue having the formula

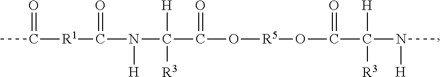

p molar equivalents of a second monomer residue having the formula

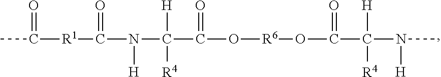

q molar equivalents of a third monomer residue having the formula

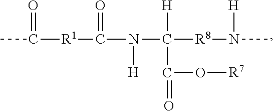

and
x molar equivalents of a fourth monomer residue having the formula

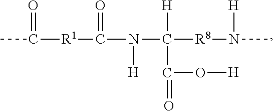

wherein m+p+q+x=1;
m is from 0 to 0.8, p is from 0 to 0.95, m+p is from is from 0.5 to 1, q is from 0 to 0.35, and x is from 0 to 0.25;
n is from 5 to 300;
R¹ is (C₂-C₂₀)alkylene;
R³ and R⁴ are selected from the group consisting of hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃⁺, —(CH₂)₃NHC(═NH₂⁺)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph—CH₂—, CH═C—CH₂—, (CH₃)₂CH—, Ph—NH—,

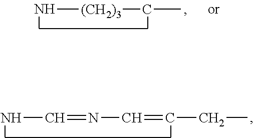

wherein R³ and R⁴ are the same or different;
R⁵ is (C₂-C₂₀)alkylene, optionally having one or more alkylene carbon atoms (—C—) replaced by an oxygen atom (—O—), and/or optionally having 5 or 6 consecutive alkylene carbon atoms together form a respective 5- or 6-membered carbocyclic ring;
R⁶ is

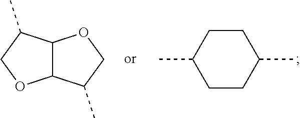

R⁷ is (C₆-C₁₀)aryl (C₁-C₆)alkylene; and
R⁸ is (C₃-C₈)alkylene.
24. The implantable glucose sensor of exemplary embodiment 23, wherein, in the structure of the PEA, p is from 0 to 0.8 and m+p is from is from 0.5 to 0.9.
25. The implantable glucose sensor of exemplary embodiment 24 or 25, wherein, in the structure of the PEA, q is from 0.05 to 0.25.
26. The implantable glucose sensor of any one of exemplary embodiments 23-25, wherein, in the structure of the PEA, x is from 0.05 to 0.25.
27. The implantable glucose sensor of any one of exemplary embodiments 23-26, wherein, in the structure of the PEA, q:x is from 9:1 to 1:4.
28. The implantable glucose sensor of any one of exemplary embodiments 23-27, wherein, in the structure of the PEA, q:x is from 4:1 to 1:4.
29. The implantable glucose sensor of any one of exemplary embodiments 23, 24 or 26, wherein, in the structure of the PEA, q is 0.
30. The implantable glucose sensor of any one of exemplary embodiments 23-25, wherein, in the structure of the PEA, x is 0.
31. The implantable glucose sensor of any one of exemplary embodiments 23 or 25-30, wherein, in the structure of the PEA, m is 0.
32. The implantable glucose sensor of exemplary embodiment 23, wherein the structure of the PEA is further characterized by one or more of the following:
m is from 0.1 to 0.5, p is from 0.1 to 0.75, m+p is from is from 0.5 to 0.8, q is from 0.1 to 0.35, and x is from 0 to 0.1;
R¹ is (C₂-C₁₀)alkylene;
R³ and R⁴ are (C₁-C₆)alkyl;
R⁵ is (C₂-C₁₀)alkylene;
R⁶ is

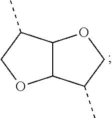

R⁷ is Ph-(C₁-C₆)alkylene; and
R⁸ is (C₃-C₆)alkylene.
33. The implantable glucose sensor of exemplary embodiment 23, wherein the structure of the PEA is further characterized by one or more of the following:
m is from 0.2 to 0.4, p is from 0.35 to 0.55, m+p is from is from 0.65 to 0.85, q is from 0.08 to 0.25, and x is from 0.03 to 0.15;
R¹ is (C₂-C₁₀)alkylene;
R³ and R⁴ are (C₁-C₆)alkyl;
R⁵ is (C₂-C₁₀)alkylene;
R⁶ is

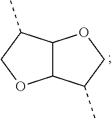

R⁷ is Ph-(C₁-C₆)alkylene; and
R⁸ is (C₃-C₆)alkylene.
34. The implantable glucose sensor of exemplary embodiment 23, wherein the structure of the PEA is further characterized by one or more of the following:
m is 0.3, p is 0.45, q is 0.25, and x is 0;
R¹ is octylene;
R³ and R⁴ are isobutyl;
R⁵ is hexylene;
R⁶ is

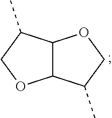

R⁷ is benzyl; and
R⁸ is butylene.
35. The implantable glucose sensor of any one of exemplary embodiments 23-34, wherein the coating has an oxygen to glucose permeability ratio of at least about 440.
36. The implantable glucose sensor of any one of exemplary embodiments 23-35, wherein the coating has a glucose permeability of at least about 10⁻¹⁵ cm²/s.
37. The implantable glucose sensor of any one of exemplary embodiments 23-36, wherein the coating has a glucose permeability is at least about 10⁻¹² cm²/s.
38. The implantable glucose sensor of any one of exemplary embodiments 23-37, wherein the PEA has a glass transition temperature in the hydrated state of less than about 37° C.
39. The implantable glucose sensor of any one of exemplary embodiments 23-38, wherein the PEA is amorphous.
40. The implantable glucose sensor of any one of exemplary embodiments 23-39, wherein the PEA has a water uptake of less than about 30 wt-% after two weeks in phosphate buffered saline (PBS) at 37° C.
41. The implantable glucose sensor of any one of exemplary embodiments 23-40, wherein the PEA has a water uptake that remains within about +/−20%, over a period from 4 days to two weeks in phosphate buffered saline (PBS) at 37° C.
42. The implantable glucose sensor of any one of exemplary embodiments 23-41, wherein the PEA in the dry state at 37° C. has a shear storage modulus G′ at a frequency of 1 Hz of at least about 0.1 MPa.
43. The implantable glucose sensor of any one of exemplary embodiments 23-42, wherein the coating does not include an anti-inflammatory agent.
44. The implantable glucose sensor of any one of exemplary embodiments 23-43, wherein the coating further comprises an additional polymer.
45. The implantable glucose sensor of exemplary embodiment 44, wherein the additional polymer underlies an external surface layer of the PEA, said external surface layer being configured for direct contact with interstitial fluid of the patient.
46. A coating for an implantable glucose sensor, said coating comprising a polyesteramide (PEA) having a structure as defined in any one of exemplary embodiments 1 to 11 and/or characterized by a property as defined in any one of exemplary embodiments 12 to 20, said coating optionally comprising an additional polymer.
47. An implantable glucose sensor having a coating as defined in exemplary embodiment 46 applied to a component of the implantable glucose sensor for penetrating the skin of a patient.
48. The implantable glucose sensor of exemplary embodiment 47, wherein the component is a needle-like structure or an insulin infusion cannula.
49. The implantable glucose sensor of any one of exemplary embodiments 23-45 wherein the coating is present on a component of the implantable glucose sensor for penetrating the skin of a patient.
50. The implantable glucose sensor of any one of exemplary embodiments 23-45 wherein the coating is present on a needle-like structure or an insulin infusion cannula.
51. A method for continuously monitoring a glucose level in a patient, the method comprising implanting the implantable glucose sensor of any one of exemplary embodiments 23-45 or 47-50 in the patient, such that glucose and oxygen in interstitial fluid of the patient pass through said coating.

1. A glucose limiting membrane for use in continuous glucose monitoring (CGM), said glucose limiting membrane comprising a polyesteramide (PEA), wherein the PEA has a structure including n units of:
m molar equivalents of a first monomer residue having the formula 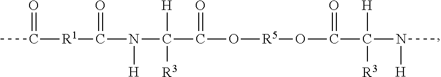
p molar equivalents of a second monomer residue having the formula 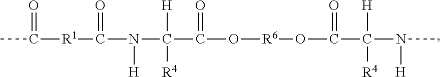
q molar equivalents of a third monomer residue having the formula 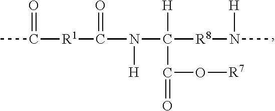
and
x molar equivalents of a fourth monomer residue having the formula 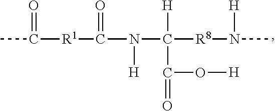
wherein m+p+q+x=1;
m is from 0 to 0.8, p is from 0 to 0.95, m+p is from is from 0.5 to 1, q is from 0 to 0.35, and x is from 0 to 0.25;
n is from 5 to 300;
R¹ is (C₂-C₂₀)alkylene;
R³ and R⁴ are selected from the group consisting of hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃⁺, —(CH₂)₃NHC(═NH₂⁺)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph—CH₂—, CH═C—CH₂—, (CH₃)₂CH—, Ph—NH—, 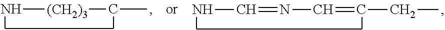
wherein R³ and R⁴ are the same or different;
R⁵ is (C₂-C₂₀)alkylene, optionally having one or more alkylene carbon atoms (—C—) replaced by an oxygen atom (—O—), and/or optionally having 5 or 6 consecutive alkylene carbon atoms together form a respective 5- or 6-membered carbocyclic ring;
R⁶ is 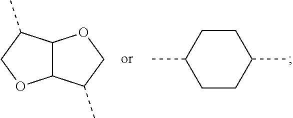
R⁷ is (C₆-C₁₀)aryl (C₁-C₆)alkylene; and
R⁸ is (C₃-C₈)alkylene.

2. The glucose limiting membrane of claim 1, wherein, in the structure of the PEA, p is from 0 to 0.8 and m+p is from is from 0.5 to 0.9.

3. The glucose limiting membrane of claim 1, wherein, in the structure of the PEA, q is from 0.05 to 0.25.

4. The glucose limiting membrane of claim 1, wherein, in the structure of the PEA, x is from 0.05 to 0.25.

5. The glucose limiting membrane of claim 1, wherein, in the structure of the PEA, q:x is from 9:1 to 1:4.

6. The glucose limiting membrane of claim 1, wherein, in the structure of the PEA, q:x is from 4:1 to 1:4.

7. The glucose limiting membrane of claim 1, wherein, in the structure of the PEA, q is 0.

8. The glucose limiting membrane of claim 1, wherein, in the structure of the PEA, x is 0.

9. The glucose limiting membrane of claim 1, wherein, in the structure of the PEA, m is 0.

10. The glucose limiting membrane of claim 1, wherein the structure of the PEA is further characterized by one or more of the following:
m is from 0.1 to 0.5, p is from 0.1 to 0.75, m+p is from is from 0.5 to 0.8, q is from 0.1 to 0.35, and x is from 0 to 0.1;
R¹ is (C₂-C₁₀)alkylene;
R³ and R⁴ are (C₁-C₆)alkyl;
R⁵ is (C₂-C₁₀)alkylene;
R⁶ is 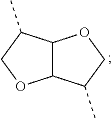
R⁷ is Ph-(C₁-C₆)alkylene; and
R⁸ is (C₃-C₆)alkylene.

11. The glucose limiting membrane of claim 10, wherein the structure of the PEA is further characterized by one or more of the following:
m is 0.3, p is 0.45, q is 0.25, and x is 0;
R¹ is octylene;
R³ and R⁴ are isobutyl;
R⁵ is hexylene;
R⁶ is 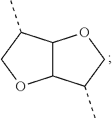
R⁷ is benzyl; and
R⁸ is butylene.

12. The glucose limiting membrane of claim 1, having an oxygen to glucose permeability ratio of at least about 440.

13. The glucose limiting membrane of claim 1, having a glucose permeability of at least about 10⁻¹⁵ cm²/s.

14. The glucose limiting membrane of claim 13, wherein the glucose permeability is at least about 10⁻¹² cm²/s.

15. The glucose limiting membrane of claim 1, wherein the PEA has a glass transition temperature in the hydrated state of less than about 37° C.

16. The glucose limiting membrane of claim 1, wherein the PEA is amorphous.

17. The glucose limiting membrane of claim 1, wherein the PEA has a water uptake of less than about 30 wt-% after two weeks in phosphate buffered saline (PBS) at 37° C.

18. The glucose limiting membrane of claim 1, wherein the PEA has a water uptake that remains within about +/−20%, over a period from 4 days to two weeks in phosphate buffered saline (PBS) at 37° C.

19. The glucose limiting membrane of claim 1, wherein the PEA in the dry state at 37° C. has a shear storage modulus G′ at a frequency of 1 Hz of at least about 0.1 MPa.

20. The glucose limiting membrane of claim 1, wherein the membrane does not include an anti-inflammatory agent.

21. The glucose limiting membrane of claim 1, further comprising an additional polymer.

22. The glucose limiting membrane of claim 21, wherein the additional polymer underlies an external surface layer of the PEA, said external surface layer being configured for direct contact with interstitial fluid of the patient.

23. An implantable glucose sensor comprising a coating comprising a polyesteramide (PEA), wherein the PEA has a structure including n units of:
m molar equivalents of a first monomer residue having the formula 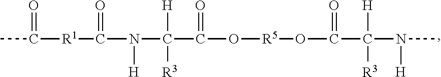
p molar equivalents of a second monomer residue having the formula 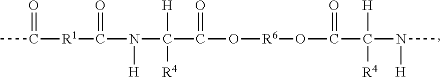
q molar equivalents of a third monomer residue having the formula 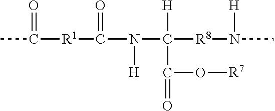
and
x molar equivalents of a fourth monomer residue having the formula 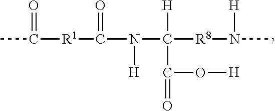
wherein m+p+q+x=1;
m is from 0 to 0.8, p is from 0 to 0.95, m+p is from is from 0.5 to 1, q is from 0 to 0.35, and x is from 0 to 0.25;
n is from 5 to 300;
R¹ is (C₂-C₂₀)alkylene;
R³ and R⁴ are selected from the group consisting of hydrogen, (C₁-C₆)alkyl, (C₂-C₆)alkenyl, (C₂-C₆)alkynyl, (C₆-C₁₀)aryl, —CH₂SH, —(CH₂)₂S(CH₃), —CH₂OH, —CH(OH)CH₃, —(CH₂)₄NH₃⁺, —(CH₂)₃NHC(═NH₂⁺)NH₂, —CH₂COOH, —CH₂—CO—NH₂, —CH₂CH₂—CO—NH₂, —CH₂CH₂COOH, CH₃—CH₂—CH(CH₃)—, (CH₃)₂CH—CH₂—, H₂N—(CH₂)₄—, Ph—CH₂—, CH═C—CH₂—, (CH₃)₂CH—, Ph—NH—, 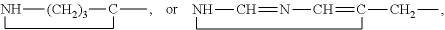
wherein R³ and R⁴ are the same or different;
R⁵ is (C₂-C₂₀)alkylene, optionally having one or more alkylene carbon atoms (—C—) replaced by an oxygen atom (—O—), and/or optionally having 5 or 6 consecutive alkylene carbon atoms together form a respective 5- or 6-membered carbocyclic ring;
R⁶ is 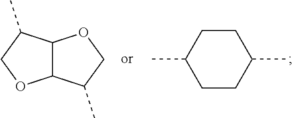
R⁷ is (C₆-C₁₀)aryl (C₁-C₆)alkylene; and
R⁸ is (C₃-C₈)alkylene.

24. The implantable glucose sensor of claim 23, wherein, in the structure of the PEA, p is from 0 to 0.8 and m+p is from is from 0.5 to 0.9.

25. The implantable glucose sensor of claim 24, wherein, in the structure of the PEA, q is from 0.05 to 0.25.

26. The implantable glucose sensor of claim 23, wherein, in the structure of the PEA, x is from 0.05 to 0.25.

27. The implantable glucose sensor of claim 23, wherein, in the structure of the PEA, q:x is from 9:1 to 1:4.

28. The implantable glucose sensor of claim 23, wherein, in the structure of the PEA, q:x is from 4:1 to 1:4.

29. The implantable glucose sensor of claim 23, wherein, in the structure of the PEA, q is 0.

30. The implantable glucose sensor of claim 23, wherein, in the structure of the PEA, x is 0.

31. The implantable glucose sensor of claim 23, wherein, in the structure of the PEA, m is 0.

32. The implantable glucose sensor of claim 23, wherein the structure of the PEA is further characterized by one or more of the following:
m is from 0.1 to 0.5, p is from 0.1 to 0.75, m+p is from is from 0.5 to 0.8, q is from 0.1 to 0.35, and x is from 0 to 0.1;
R¹ is (C₂-C₁₀)alkylene;
R³ and R⁴ are (C₁-C₆)alkyl;
R⁵ is (C₂-C₁₀)alkylene;
R⁶ is 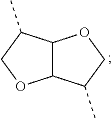
R⁷ is Ph-(C₁-C₆)alkylene; and
R⁸ is (C₃-C₆)alkylene.

33. The implantable glucose sensor of claim 23, wherein the structure of the PEA is further characterized by one or more of the following:
m is from 0.2 to 0.4, p is from 0.35 to 0.55, m+p is from is from 0.65 to 0.85, q is from 0.08 to 0.25, and x is from 0.03 to 0.15;
R¹ is (C₂-C₁₀)alkylene;
R³ and R⁴ are (C₁-C₆)alkyl;
R⁵ is (C₂-C₁₀)alkylene;
R⁶ is 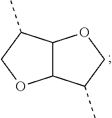
R⁷ is Ph-(C₁-C₆)alkylene; and
R⁸ is (C₃-C₆)alkylene.

34. The implantable glucose sensor of claim 23, wherein the structure of the PEA is further characterized by one or more of the following:
m is 0.3, p is 0.45, q is 0.25, and x is 0;
R¹ is octylene;
R³ and R⁴ are isobutyl;
R⁵ is hexylene;
R⁶ is 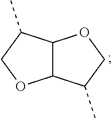
R⁷ is benzyl; and
R⁸ is butylene.

35. The implantable glucose sensor of claim 23, wherein the coating has an oxygen to glucose permeability ratio of at least about 440.

36. The implantable glucose sensor of claim 23, wherein the coating has a glucose permeability of at least about 10⁻¹⁵ cm²/s.

37. The implantable glucose sensor of claim 23, wherein the coating has a glucose permeability is at least about 10⁻¹² cm²/s.

38. The implantable glucose sensor of claim 23, wherein the PEA has a glass transition temperature in the hydrated state of less than about 37° C.

39. The implantable glucose sensor of claim 23, wherein the PEA is amorphous.

40. The implantable glucose sensor of claim 23, wherein the PEA has a water uptake of less than about 30 wt-% after two weeks in phosphate buffered saline (PBS) at 37° C.

41. The implantable glucose sensor of claim 23, wherein the PEA has a water uptake that remains within about +/−20%, over a period from 4 days to two weeks in phosphate buffered saline (PBS) at 37° C.

42. The implantable glucose sensor of claim 23, wherein the PEA in the dry state at 37° C. has a shear storage modulus G′ at a frequency of 1 Hz of at least about 0.1 MPa.

43. The implantable glucose sensor of claim 23, wherein the coating does not include an anti-inflammatory agent.

44. The implantable glucose sensor of claim 23, wherein the coating further comprises an additional polymer.

45. The implantable glucose sensor of claim 44, wherein the additional polymer underlies an external surface layer of the PEA, said external surface layer being configured for direct contact with interstitial fluid of the patient.

46. A coating for an implantable glucose sensor, said coating comprising a polyesteramide (PEA) having a structure as defined in claim 1 and/or characterized by a property as defined in said coating optionally comprising an additional polymer.

47. An implantable glucose sensor having a coating as defined in claim 46 applied to a component of the implantable glucose sensor for penetrating the skin of a patient.

48. The implantable glucose sensor of claim 47, wherein the component is a needle-like structure or an insulin infusion cannula.

49. The implantable glucose sensor of claim 23 wherein the coating is present on a component of the implantable glucose sensor for penetrating the skin of a patient.

50. The implantable glucose sensor of claim 23 wherein the coating is present on a needle-like structure or an insulin infusion cannula.

51. A method for continuously monitoring a glucose level in a patient, the method comprising implanting the implantable glucose sensor of claim 23 in the patient, such that glucose and oxygen in interstitial fluid of the patient pass through said coating.
